(19) United States
(12) Patent Application Publication
(10) Pub. No.: US 2012/0184820 A1
(43) Pub. Date: Jul. 19, 2012

(54) ENDOSCOPE WITH ADJUSTABLE VIEWING ANGLE

(76) Inventors: Jan Dahmen, Seitingen-Oberflacht (DE); Fang Lei, Durchhausen (DE)

(21) Appl. No.: 13/326,088

(22) Filed: Dec. 14, 2011

(30) Foreign Application Priority Data
Dec. 16, 2010 (DE) 10 2010 063 230.9

(51) Int. Cl. A61B 1/07

(52) U.S. Cl. 600/173

(57) ABSTRACT

An endoscope with adjustable viewing angle includes a light conductor with a light inlet surface and a light outlet surface to transmit illuminating light from the light inlet surface to the light outlet surface of the light conductor. The light conductor can pivot with the light inlet surface and light outlet surface around a pivot axis in relation to the endoscope.

### CROSS-REFERENCE TO RELATED APPLICATIONS

The present application claims priority of German patent application No. 10 2010 063 230.9 filed on Dec. 16, 2010, the content of which is incorporated herein by reference.

### FIELD OF THE INVENTION

The present invention relates to an endoscope with an adjustable viewing angle.

### BACKGROUND OF THE INVENTION

In addition to endoscopes for medical and non-medical technical applications, whose viewing angle is parallel to the longitudinal axis of the endoscope shaft, endoscopes with other fixed viewing angles have been developed for some time. The viewing angle of an endoscope is understood here and hereinafter always to mean the direction facing from the distal end of the endoscope, in which an object is situated that appears in the center of the image recorded by means of the endoscope. In many applications, however, a fixed viewing angle is a disadvantage. In the worst case, for example during a medical procedure, the endoscope must be replaced numerous times. In such cases it is an advantage to use an endoscope with a viewing angle that can be selected or adjusted in situ.

Observing an object in a cavity by means of an endoscope assumes as a rule that there is some illumination of the object. For this purpose an endoscope comprises, for example, lightwave conductors, in particular glass fibers, by means of which an illuminating light is transmitted from the proximal end of the endoscope along the shaft to the distal end of the endoscope. Light outlet surfaces of the lightwave conductors on the distal end of the endoscope are positioned and configured in such a way that the entire visual field or viewing field is sufficiently illuminated.

In an endoscope with adjustable viewing angle, the illuminating light on the distal end of the endoscope, in the simplest case, is distributed in such a way that the entire visual field is illuminated independently of the particular viewing angle selected. This results, however, in a series of disadvantages. In particular, light capacity is wasted, because the entire visual fields of all selectable viewing angles are illuminated constantly, independently of the viewing angle that is actually selected. Thus, at a predetermined desired brightness, a markedly higher lighting capacity must be provided altogether than with an endoscope with a fixed viewing angle.

An additional disadvantage arises from the fact that illuminating light of high intensity can photothermally or photochemically damage tissue or other objects. With an endoscope with fixed viewing angle, the distal end of the endoscope is at too close a distance to an object, at least on observing the recorded image. In using a video camera on the endoscope, an automatic warning of users is also possible if the brightness of a recorded image exceeds a predetermined threshold. With an endoscope with adjustable viewing angle, however, part of the illuminating light impinges on objects lying outside the visual field. Therefore there is no undesired approach of the distal end of the endoscope to these objects, and no resulting illumination of these objects with too high a radiant capacity.

A further disadvantage consists in the fact that illuminating light radiated outside the visual field can also be dispersed or reflected by objects or opaque media. The reflected or dispersed illuminating light can arrive directly or indirectly in the observation beam path. Consequently, contrasts and the distinguishability of objects, especially in dark image areas, can be reduced.

An additional disadvantage comes from the fact that the illuminating intensity or intensity of the illuminating light is essentially constant in the direction in which the viewing angle can be varied (often referred to also as the vertical direction), while it decreases slightly as a rule toward the edge of the visual field in the direction perpendicular thereto (often also called the horizontal direction). However, users of endoscopes with fixed viewing angle are as a rule accustomed to an illuminating intensity that slightly declines toward the edge of the visual field both in the horizontal and in the vertical directions. The illuminating intensity that is constant in the vertical direction can therefore be experienced as an irritant.

Patent application DE 600 15 375 T2 describes an arrangement of several prisms. One of the prisms can be rotated around an axis in order to cast illuminating light at an adjustable viewing angle. The inventors of the present invention, however, have determined that in the described arrangement of prisms the distribution of the illuminating light inside the visual field is insufficient in many cases. In addition, with the arrangements described in DE 600 15 375 T2, it can be costly in practice to achieve simultaneously a largely optical isolation of the illumination and observation beam paths, a high light intensity in the observation beam path, low losses in the illumination beam path and a small shaft diameter.

### SUMMARY OF THE INVENTION

An object of the present invention consists in providing an improved endoscope with adjustable viewing angle.

This object is achieved through the contents of the independent claims.

Refinements are indicated in the dependent claims.

Embodiments of the present invention are based on the idea of providing one or more pivotable light conductors on the distal end of an endoscope with adjustable viewing angle and correspondingly adjustable illuminating angle, to conduct or transmit illuminating light. Said light conductor or light conductors are pivoted together with their light inlet surface and their light outlet surface around a pivot axis and can be curved as desired between the light inlet surface and the light outlet surface within the limits provided in light conductors, in order to transmit the illuminating light with small losses at reduced space requirements.

An endoscope with adjustable viewing angle includes a light conductor with a light inlet surface and a light outlet surface for transmitting illuminating light from the light inlet surface to the light outlet surface of the light conductor, whereby the light conductor with the light inlet surface and light outlet surface can be pivoted around a pivot axis in relation to the endoscope.

The endoscope is configured in particular to pivot the viewing angle and the angle of illumination together. The viewing angle is the angle at which an object is situated with reference to the distal end of the endoscope, said object appearing in the center of a recorded image upon observation through the endoscope. The angle of illumination is the center direction in which the illuminating light is radiated with reference to the distal end of the endoscope. The angle of illumination and the viewing angle, in particular, correspond to one another. Alternatively, the viewing angle and the angle of illumination can be adjusted independently of one another or the angle of illumination can be varied in relation to the viewing angle within certain boundaries, for instance to achieve a complete illumination of the observed area for objects at small distances.

The endoscope viewing angle, in particular, can be pivoted around a pivot axis that is perpendicular to the longitudinal axis of the shaft of the endoscope. The endoscope longitudinal axis is, in particular, the longitudinal axis of the shaft. In the case of a rigid, straight shaft, the longitudinal axis of the shaft is the straight line on which the center points of the cross-section surfaces of the shaft are situated. In the case of a flexible shaft, the endoscope longitudinal axis is the longitudinal axis of the distal end of the shaft, that is, the straight line on which the center points of the cross-section surfaces of the shaft are situated close to its distal end.

Illuminating light that is to be transmitted or guided by the pivotable light conductor can be transmitted by means of a fixed light conductor from a light source that is positioned on the proximal end of the endoscope or coupled by a light conductor cable with the proximal end of the endoscope to the distal end of the endoscope, where it can then be switched into the light inlet surface of the pivotable light conductor. Alternatively, the pivotable light conductor transmits illuminating light generated from a light source positioned on the distal end of the endoscope.

The pivot axis of the pivotable light conductor is, in particular, the pivot axis of the viewing angle or is parallel to it. A lens or other optic element that forms the illuminating light beam can be positioned on the light outlet surface of the pivotable light conductor. Alternatively, the light outlet surface of the pivotable light conductor itself can be curved in order to form the illuminating light beam. The endoscope can include one, two, or more pivotable light conductors, which can be pivotable together or independently of one another.

Light conductors make possible a transmission of illuminating light with a high degree of efficiency or with low losses also along pathways that are of simple or multiple curvature and that otherwise would require several reflections on reflecting surfaces. The spatial conditions on the distal end of an endoscope with adjustable viewing angle are restricted as a rule and limited primarily by the observation beam path and the cross-section of the shaft that is available. The observation beam path must be laid out in such a way that the greatest possible amount of light that is reflected, dispersed or emitted by the observed object is captured and steered with the highest possible imaging capacity onto a light-sensitive sensor or is switched into a rod lens system or an arranged bundle of lightwave conductors. The use of one or more light conductors for transmitting illuminating light makes possible a particular flexibility in configuring the distal end of the endoscope and the observation beam path.

In an endoscope as described here, the pivotable light conductor is, in particular, rigid.

A rigid or bend-resistant light conductor constitutes, especially at a small length, a robust component that is low in wear and also can transmit illuminating light in a manner both reliable and low in losses even after long use and numerous changes in the illuminating angle. In particular, it becomes possible to avoid the disadvantages of a flexible light conductor in which the breaking of individual fibers or other consequences of material fatigue can gradually increase losses in transmitting illuminating light.

The rigid pivotable light conductor includes, in particular, a bundle of lightwave conductors that are cemented, melded or cast together in order to stiffen the bundle.

A bundle or several bundles of glass or plastic lightwave conductors make it possible to transmit illuminating light reliably and with low loss and can be put into just about any desired shape before the partial or complete cementing, melding or casting. The individual lightwave conductors are, for example, multi-mode or mono-mode fibers with an index of refraction that is continual or varying by stages inside the cross-section. Owing to cementing, melding or casting, the bundle can acquire a high mechanical robustness. Damage to individual lightwave conductors is thereby prevented or at least made less likely.

An endoscope as described here can include a pivotable reflecting surface for diverting illuminating light from a direction parallel to the pivot axis of the pivotable light conductor to the light inlet surface of the pivotable light conductor, such that the pivotable reflecting surface is designed and configured in order to be pivoted together with the pivotable light conductor.

The reflecting surface is, in particular, a prism surface or other transparent body surface that is reflecting on the basis of total reflectance or of mirroring, and in said body the illuminating light spreads out between a light inlet surface and a light outlet surface. The surface normal of the light inlet surface of the pivotable light conductor is, in particular, perpendicular or essentially perpendicular to the pivot axis of the light conductor. The pivotable light conductor can comprise a straight or essentially straight form or a comparatively low curvature, owing to the diversion of the illuminating light by the reflecting surface. The reflecting surface can be configured in such a way that it demands little structural space.

The pivotable reflecting surface is, in particular, a reflecting surface of a pivotable prism with a light inlet surface and light outlet surface, such that the light inlet surface of the light conductor is joined with the light outlet surface of the prism.

A pivotable prism in the sense of the present invention is a pivotable transparent body that comprises the shape of a prism in the strict geometric sense, the surface of which therefore includes two equal and parallel polygons whose corresponding corners are connected together by parallel lateral edges. In addition the term “prism” in optics is used for transparent bodies with a light inlet surface and light outlet surface that are not parallel to one another.

In the present invention, the transparent body can even more commonly comprise a flat or curved light inlet surface, a flat or curved reflecting surface and a flat or curved light outlet surface, for diverting illuminating light. A curved light inlet surface, curved reflecting surface and/or a curved light outlet surface can cause a change in the divergence or convergence of the illuminating light or another beam formation.

The light inlet surface of the pivotable light conductor and the light outlet surface of the transparent body are, in particular, cemented or welded.

A prism or other transparent body can constitute a compact and robust optic element for diverting illuminating light.

In an endoscope as described here, the pivotable light conductor can comprise a curvature.

In particular, a curvature at an angle of 90 degrees or essentially 90 degrees can constitute an alternative to the aforementioned pivotable reflecting surface, in order to divert illuminating light from a direction parallel to the pivot axis of the pivotable light conductor into the angle of illumination. For this purpose the light conductor, in particular, comprises on its light inlet surface a direction parallel to the pivot axis and on its light outlet surface a direction parallel to the angle of illumination. In a light conductor that in modifying the angle of illumination is neither elastically nor structurally deformed, small curvature radii can also be achieved with small transmission losses and high reliability.

The pivotable light conductor is positioned on the light inlet surface of the pivotable light conductor, in particular in a direction parallel to a pivot axis of the pivotable light conductor.

In an endoscope in which the pivotable light conductor is positioned on the light inlet surface of the pivotable light conductor in a direction parallel to the pivot axis of the pivotable light conductor, the light inlet surface of the pivotable light conductor, in particular, has the shape of a circle or circular ring.

A light inlet surface of the pivotable light conductor that is circular or circular-ring-shaped and, in particular, symmetrical to the pivot axis of the pivotable light conductor makes possible a switching of illuminating light into the pivotable light conductor independently of the position of the pivotable light conductor. The illumination of the visual field can thereby be independent of the viewing angle. This applies in particular when a correspondingly configured and positioned light outlet surface of a light source or of a fixed light conductor is situated opposite the circular or circular-ring-shaped light inlet surface of the pivotable light conductor.

A circular-ring-shaped configuration of the light inlet surface of the pivotable light conductor and of the light outlet surface of the light source or of the fixed light conductor makes possible, for example, an arrangement of a bearing, shaft, or portion of the observation beam path inside the circular-ring-shaped light inlet or light outlet surface and thus an especially compact arrangement.

An endoscope as described here can include a fixed reflecting surface to divert illuminating light in a direction parallel to the pivot axis of the pivotable light conductor.

The fixed reflecting surface is, in particular, a reflecting surface of a mirror or prism or of another transparent body that reflects on the basis of total reflectance or of a reflecting coating. For example, illuminating light that is transmitted to the distal end of the endoscope by means of a fixed light conductor is diverted in a direction parallel to the pivot axis of the pivotable light conductor by means of a fixed reflecting surface. A fixed reflecting surface can make possible an especially compact diversion of illuminating light.

An endoscope as described here can include a fixed light conductor to transmit illuminating light to the distal end of the endoscope, such that the fixed light conductor comprises a curvature near its light outlet surface.

The surface normal of the light outlet surface of the fixed light conductor is, in particular, parallel to the pivot axis of the pivotable light conductor. The light outlet surface of the fixed light conductor is, in particular, positioned opposite a light inlet surface of the pivotable light conductor that corresponds in terms of its shape, arrangement, and placement. The fixed light conductor is curved, in particular, by 90 degrees near its light outlet surface.

The fixed light conductor, curved on its distal end, can make possible a structurally especially simple and low-loss transmission of illuminating light to the pivotable light conductor. Because the fixed light conductor is not deformed in a modification of the viewing angle and angle of illumination, but instead only the pivotable light conductor is pivoted with respect to the fixed light conductor, it is possible to achieve a small curvature radius on the fixed light conductor.

An endoscope as described here can comprise a light shaft, which can be pivoted around the pivot axis of the viewing angle, to optically separate the observation beam path from the illumination beam path, whereby the light outlet surface of the pivotable light conductor is positioned outside on the light shaft.

The light shaft has, in particular, the shape of a truncated cone with circular or non-circular base surface, in particular of a truncated cone or pyramid, with a circular, elliptical or right-angle edge. The light outlet surface of the pivotable light conductor, in particular, is positioned in one or more strip-shaped parts, each essentially straight or arched, outside on the edge of the light shaft.

A light shaft pivoting with the viewing angle makes possible an extensive or complete optic isolation or separation of the observation beam path from the illumination beam path. A positioning of the light outlet surface in one or more parts outside in the light shaft ensures in structural, simple manner that the light outlet surface and thus also the angle of illumination pivot with the viewing angle. Simultaneously, the light outlet surface can be positioned especially close to the observation beam path in order to minimize disturbing shadows.

In an endoscope as described here, a light outlet device can be positioned on the light outlet surface of the pivotable light conductor. The light outlet device can include one or more lenses, mirrors, prisms or other optic elements for forming an illuminating light bundle uncoupled from the light conductor on the distal end. Alternatively, the light outlet surface of the pivotable light conductor is configured for forming the uncoupled illuminating light bundle. In the simplest case the light outlet surface of the pivotable light bundle is flat, and the divergence of the exiting illuminating light bundle is established on the basis of the transmission properties of the light conductor immediately in switching the illuminating light into the pivotable light conductor.

### BRIEF DESCRIPTION OF THE DRAWINGS

Embodiments are described in greater detail hereinafter with reference to the appended drawings, which are as follows:

FIG. 1 shows a schematic depiction of an endoscope with adjustable viewing angle.

FIG. 2 shows a schematic depiction of the distal end of an embodiment of the endoscope from FIG. 1.

FIG. 3 shows another schematic depiction of the distal end from FIG. 2.

FIG. 4 shows schematic depictions of three variants of the embodiment from FIGS. 2 and 3.

FIG. 5 shows schematic depictions of two variants of the embodiment from FIGS. 2 and 3.

FIG. 6 shows a schematic depiction of the distal end of an additional embodiment of the endoscope from FIG. 1.

FIG. 7 shows a schematic depiction of the distal end of an additional embodiment of the endoscope from FIG. 1.

FIG. 8 shows a schematic depiction of the distal end of an additional embodiment of the endoscope from FIG. 1.

FIG. 9 shows a schematic depiction of the distal end of an additional embodiment of the endoscope from FIG. 1.

FIG. 10 shows a schematic depiction of a device for pivoting.

FIG. 11 shows an additional schematic depiction of the device from FIG. 10.

### DETAILED DESCRIPTION OF THE INVENTION

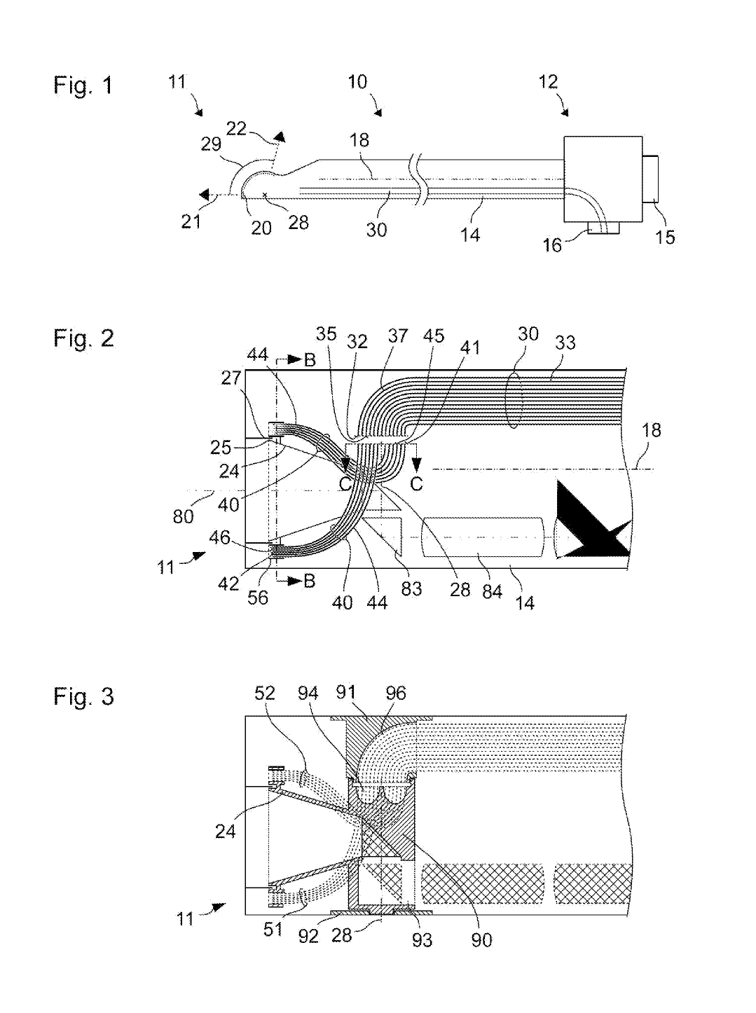

FIG. 1 shows a schematic depiction of an endoscope 10 with a distal end 11, a proximal end 12, and a rigid shaft 14 that extends from the distal end 11 to the proximal end 12. Alternatively, the shaft 14 is flexible or partly flexible. The cross-section of the shaft 14 or at least the outer contour of the cross-section of the shaft 14 is constant or essentially constant between the distal end 11 and the proximal end 12. In particular, the contour of the cross-section of the shaft 14 is circular or elliptical in shape. In this case the longitudinal axis 18 of the endoscope 10 illustrated in FIG. 1 is the axis of symmetry of the mantle surface of the shaft 14 between the distal end 11 and the proximal end 12. In a cylindrical mantle surface of the shaft 14, the longitudinal axis 18 is also the sum of the center points or surface centers of gravity of the cross-sections of the shaft 14 between the distal end 11 and the proximal end 12. In a cylindrical mantle surface of the shaft 14, the longitudinal axis 18 is also the axis of symmetry of the mantle surface.

On the distal end 11, the shape of the shaft 14 departs from the cylindrical symmetry as is depicted by way of example in FIG. 1. In particular, the shaft 14 comprises on the distal end 11 an opening that is locked by a transparent window component 20 with a vaulted surface. In particular, the window component 20 closes the opening with a hermetic sealing. The surface of the window component 20 has, for example, the shape of a portion of a cylindrical mantle, whereby the axis of symmetry of the cylinder is perpendicular to the longitudinal axis 18 of the endoscope 10 and to the plane of projection of FIG. 1. Alternatively, the surface of the transparent window component 20 has the shape of a portion of a spherical surface or of a rotation-symmetrical or non-rotation-symmetrical ellipsoid.

On the distal end 11 of the endoscope 10, optical devices are positioned in the shaft 14 that hereinafter are described in part with reference to FIGS. 2 through 9. These optical properties make possible a variation of the viewing angle of the endoscope between a first extreme viewing angle 21 and a second extreme viewing angle 22. The viewing angle can pivot between the two extreme viewing angles 21, 22 around a pivot axis 28 that is perpendicular to the plane of projection of FIG. 1. The viewing angle in each case is the direction based on the distal end 11 of the endoscope 10 in which an object is situated that appears in the center of an image recorded by means of the endoscope 10.

In the example illustrated in FIG. 1, the first extreme viewing angle 21 is parallel or essentially parallel to the longitudinal axis 18 of the endoscope 10. Situated between the extreme viewing angles 21, 22 is an angle area 29 that comprises approximately 120 degrees in the illustrated example. Within this angle area, the viewing angle of the endoscope 10 can be displaced or adjusted, in particular, continually.

On the proximal end 12 the endoscope 10 comprises a first coupling 15 for optically coupling the endoscope 10 with a video camera or an eyepiece, as well as a second coupling 16 to couple the endoscope 10 with a light source via a light conductor cable. One or more light conductors 30 lead from the second coupling 16 through the shaft 14 to the distal end 11 of the endoscope 10. Illuminating light generated from a light source can be transmitted to the distal end 11 of the endoscope 10 via a light conductor cable, the second coupling 16, and the light source or sources 30.

FIGS. 2 and 3 show schematic depictions of an embodiment of the distal end 11 of the endoscope 10 described above with reference to FIG. 1. FIG. 2 shows a schematic depiction whose plane of projection is perpendicular to the plane of projection of FIG. 1 and parallel to the longitudinal axis 18 of the endoscope 10 and to the pivot axis 28 of the viewing angle.

The light conductor 30 already indicated in FIG. 1 comprises a light outlet surface 32 on the distal end 11 of the endoscope 10. The light conductor 30 includes a bundle of lightwave conductors 33, whose light outlet surfaces 35 are positioned in a plane and together form the light outlet surface 32 of the light conductor 30. Immediately upstream of the light outlet surface 32 in the light path, the light conductor 30 comprises a curvature 37 by an angle of approximately 90 degrees. Upstream of the curvature 37 in the light path, the light conductor runs parallel or essentially parallel to the longitudinal axis 18 of the shaft 14 of the endoscope 10. At the light outlet surface 32 of the fixed light conductor 30, the lightwave conductors 33 and thus the entire light conductor 30 run essentially parallel to the pivot axis 28 of the viewing angle. The surface normals of the light outlet surface 32 of the light conductor 30 and of the light outlet surfaces 35 of the lightwave conductors 33 are also parallel or essentially parallel to the pivot axis 28 of the viewing angle.

In addition, pivotable light conductors 40 are positioned on the distal end 11 of the endoscope 10. Each pivotable light conductor 40 includes a light inlet surface 41, which is opposite the light outlet surface 32 of the fixed light conductor 30, and a light outlet surface 42. Every pivotable light conductor 40 includes a bundle of lightwave conductors 44. Light inlet surfaces 45 of the lightwave conductors 44 together make up the light inlet surface 41 of the pivotable light conductor. Light outlet surfaces 46 of the lightwave conductors together make up the light outlet surface 42 of the pivotable light conductor 40.

Also positioned on the distal end 11 of the endoscope 10 is the end of an observation beam path 80 upstream in the light path whose optical axis is indicated by a series of dots and dashes. The observation beam path 80, downstream of the window component illustrated in FIG. 1, includes a pivotable prism 82, a fixed prism 83, and a rod lens arrangement 84 to transmit light emanating from an object that is to be observed to the proximal end 12 of the endoscope 10. A light shaft 24 is positioned between the window component 20 that is not illustrated in FIG. 2 and the pivotable prism 82. The wall of the light shaft 24 comprises a light-absorbent material. The light shaft 24 has, for example, the shape of a truncated cone, in particular of a truncated pyramid. Close to the edge 25 of the light shaft 24 that is upstream in the light path, one or more mountings 56 are positioned for the light outlet surfaces 42 of the pivotable light conductors 44. A diaphragm 27 engages in an intermediate space between the edge 25 of the light shaft 24 and the mounting or mountings 56.

The pivotable light conductors 40 are curved between their light inlet surfaces 41 and their light outlet surfaces 42. In particular, the pivotable light conductors 40 each include one bend of at least 90 degrees. On their light inlet surfaces 41, the light conductors 40 run essentially parallel to the pivot axis 28 of the viewing angle. On their light outlet surfaces 42, the pivotable light conductors 40 run essentially parallel to the viewing angle and thus perpendicular to the pivot axis 28. The example illustrated in FIG. 2 includes two light conductors, whose light outlet surfaces 42 are positioned on opposite sides of the light shaft 24.

The pivotable light conductors 40, the pivotable prisms 82, the light shaft 24, and the mounting or mountings 56 are rigidly connected with one another and can pivot together around the pivot axis 28 of the viewing angle. This ensures that the angle of illumination essentially indicated by the arrangement of the light outlet surfaces 42 of the pivotable light conductors 40 and the viewing angle essentially indicated by the position of the pivotable prism 82 are modified together and, in particular, are always equal.

The light shaft 24 and the, in particular, round-arched diaphragm 27 largely or completely prevent the switching of illuminating light emerging from the light outlet surfaces 42 of the pivotable light conductors 40 into the observation beam path. The light outlet surface 32 of the fixed light conductor 30 and the light inlet surfaces 41 of the pivotable light conductors 40 are each essentially flat and are positioned at a small distance opposite one another. The distance between the light outlet surface 32 of the fixed light conductor 30 and the light inlet surface 41 of the pivotable light conductor is, in particular, substantially smaller than is schematically indicated in FIG. 2. Because the surface normals of the light outlet surface 32 of the fixed light conductor 30 and the light inlet surfaces 41 of the pivotable light conductors 40 are each parallel to the pivot axis 28 of the viewing angle, this applies independently or largely independently of the momentarily selected illumination and viewing angle.

The configuration of the fixed light conductor 30 and of the pivotable light conductors 40, from a bundle of lightwave conductors 33, 44 in each case, allows for small radii of curvature and a compact arrangement, in particular, of the pivotable light conductors 40. It thereby becomes possible to achieve a smaller cross-section of the shaft 14 and/or greater cross-sections in the observation beam path 80.

FIG. 3 shows a schematic sectional depiction of the embodiment described above with reference to FIG. 2. The sectional plane of FIG. 3 is parallel to the plane of projection of FIG. 2. The fixed and pivotable light conductors 30, 40 already described above with reference to FIG. 2 and the elements 82, 83, 84 of the observation beam path 80 are only indicated in broken lines. Instead, a pivotable illumination and observation device 90, not illustrated in FIG. 2, is shown.

The pivotable illumination and observation device 90 is mounted so that it can pivot around the pivot axis 28 of the viewing angle in two bearings 91, 92 secured on opposite sides of the shaft 14 of the endoscope 10. The pivotable illumination and observation device 90 includes the light shaft 24 or its wall and the mountings 56 positioned on the edge 25 of the light shaft 24. The pivotable illumination and observation device 90 can be of one-piece construction.

The pivotable prism 82 of the observation beam path 80 is held on or in the pivotable illumination and observation device 90. In addition, the pivotable illumination and observation device includes 90 channels 94 in which the pivotable light conductors 40 partly extend. These channels 94 are truncated by the sectional plane of FIG. 3. The fixed prism 83 of the observation beam path 80, which is not moved with the pivotable illumination and observation device 90, is positioned in a recess 93 of the pivotable illumination and observation device 90.

In the first bearing 91 a curved channel 96 is provided in which the fixed light conductor 30 is partly positioned. The fixed light conductor 30 can be cemented or cast or joined in other manner with the first bearing 91. The first bearing 91 and second bearing 92 can be constructed in one piece, soldered, screwed, or cemented with the wall of the shaft 14 of the endoscope 10.

The pivotable illumination and observation device 90, together with the pivotable light conductors 40 and the pivotable prism 82, forms a compact and robust component that can be pre-mounted outside the endoscope 10 and then can be inserted into it. Because the pivotable light conductors 40 are not deformed upon the pivoting of the illumination and viewing angle around the pivot axis 28, the risk of damage, in particular a break, of individual lightwave conductors 44 is low. The individual lightwave conductors 44 of the pivotable light conductors 40 are, in particular, cemented, melded or cast to one another and/or with the pivotable illumination and observation device 90 for additional improvement in robustness.

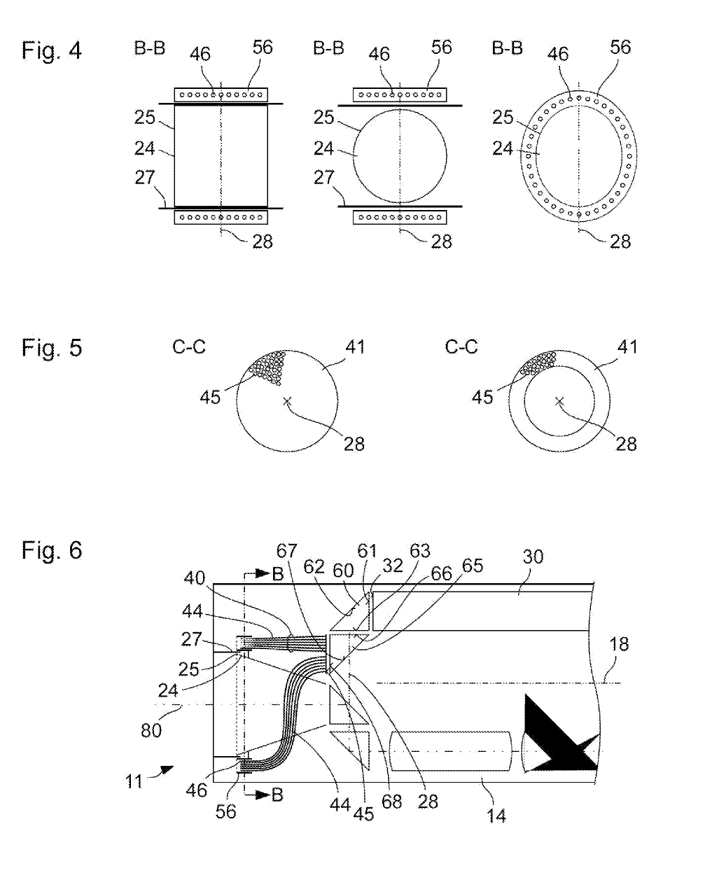

FIG. 4 shows schematic sectional depictions of three variants of the light shaft 24 and mounting or mountings 56. The location of each illustrated sectional plane B-B is indicated in FIG. 2. The sectional plane B-B is perpendicular to the plane of projection of FIGS. 1 through 3, perpendicular to the longitudinal axis 18 of the shaft 14 of the endoscope 10, and parallel to the pivot axis 28 of the illumination and viewing angle. Also indicated in each case in FIG. 4 is the position of the pivot axis 28 of the illumination and viewing angle, which however is not situated in the illustrated sectional planes.

In the example illustrated in FIG. 4 at left, the light shaft 24 has the shape of a truncated cone with rectilinear base surface and the edge 25 correspondingly has a quadrilateral form. On two sides, opposite one another, of the quadrilateral formed by the edge 25, a strip-shaped or narrow and elongated mounting 56 is positioned in each case for the ends and light outlet surfaces 46 of the lightwave conductors 44. Between the edge 25 of the light shaft 24 and the mounting 56, one of the diaphragms 27 already illustrated in FIGS. 2 and 3 is positioned in each case. Instead of two mountings on opposite facing sides of the quadrilateral formed by the edge 25, a single straight, strip-shaped mounting, for example, can be provided on one side, and a straight strip-shaped mounting or a frame-shaped mounting that surrounds the light shaft 24 on every side.

The example illustrated in the middle of FIG. 4 is distinguished from the example shown at the left in that the light shaft has the shape of a truncated cone with a circular base surface. The edge 25 of the light shaft 24 correspondingly is circular in shape.

Illustrated at the right in FIG. 4 is an example in which the light shaft 24 has the shape of a truncated cone with elliptical base surface and the edge 25 of the light shaft 24 correspondingly has the shape of an ellipse. The mounting 56 surrounds the entire light shaft 24 or its edge 25 in the form of an ellipse. Not shown in the example illustrated at the right in FIG. 4 is a diaphragm engaging between the edge 25 of the light shaft 24 and the mounting 56 such as is depicted in the examples in FIG. 4 at left and in the middle and as in FIGS. 2 and 3. Instead, the edge 25, for example, is brought up to the smallest possible distance to the window component 20 illustrated above in FIG. 1.

Instead of a truncated conical form, the light shaft 24 can have a different shape. For example, the light shaft 24 can comprise a rectangular cross-section on its upstream side in the light path and a quadrilateral cross-section on its downstream side in the light path, or an elliptical cross-section on its upstream side in the light path and a rectangular cross-section on its downstream side in the light path.

FIG. 5 shows a schematic sectional depiction of two variants of the light inlet surface 41 or of the pivotable light conductors 40. The position of the illustrated sectional plane C-C is indicated in FIG. 2. The sectional plane C-C contains in each case the light outlet surface 41. Also indicated in each case in FIG. 5 is the position of the pivot axis 28 of the illumination and viewing angle that is perpendicular to sectional plane C-C and to the light inlet surface 41. Only one part of the light inlet surfaces 45 of the lightwave conductors 44 is indicated, by way of example.

In the example illustrated at the left in FIG. 5 the light inlet surface 41 is circular, while in the example shown to the right in FIG. 5 the light inlet surface 41 is of circular ring shape. In both cases the light inlet surface 41 is formed by the light inlet surfaces 45 of the individual lightwave conductors, as mentioned above in connection with FIG. 2. In the circular-ring-shaped light inlet surface 41 illustrated to the right in FIG. 5, contrary to the depiction in FIG. 3, the pivotable illumination and observation device 90 can be mounted inside the light inlet surface 41.

FIG. 6 shows a schematic depiction of the distal end 11 of another embodiment of the endoscope 10 from FIG. 1. The embodiment in FIG. 6 resembles in some characteristics the embodiment in FIGS. 2 and 3. Contrary to the embodiment in FIGS. 2 and 3, the fixed light conductor 30 comprises no curvature close to its light outlet surface 32. Instead, the fixed light conductor 30 is, in particular, straight. The light outlet surface 32 is optically coupled with a light inlet surface 61 of a fixed prism 60. In particular, the light outlet surface 32 of the fixed light conductor 30 and the light inlet surface 61 of the fixed prism 60, contrary to the depiction in FIG. 6, are directly soldered together or are connected by a transparent cement or a transparent welding material.

The fixed prism 60 comprises a reflecting surface 62 and a light outlet surface 63. The surface normal of the light inlet surface 61 of the fixed prism 60 is parallel or essentially parallel to the longitudinal axis 18 of the shaft 14 of the endoscope 10. The surface normal of the light outlet surface 63 of the fixed prism 60 is parallel or essentially parallel to the pivot axis 28 of the illumination and viewing direction. The surface normal of the reflecting surface 62 of the fixed prism 60 is the angle-bisector or essentially the angle-bisector of the surface normals of the light inlet surface 61 and of the surface normals of the light outlet surface 62 of the fixed prism 60.

A light inlet surface 66 of a pivotable prism 65 is positioned opposite and parallel to the light outlet surface 63 of the fixed prism 60. Thus, in particular, the surface normal of the light inlet surface 66 of the pivotable prism 65 is also parallel or essentially parallel to the pivot axis 28 of the illumination and viewing angle. The pivotable prism 65, in addition, comprises a reflecting surface 67 and a light outlet surface 68. The surface normal of the light outlet surface 68 is perpendicular or essentially perpendicular to the pivot axis 28 of the illumination and viewing angle. The surface normal of the reflecting surface 67 of the pivotable prism 65 is the angle-bisector or essentially the angle-bisector of the surface normals of the light inlet surface 66 and of the surface normals of the light outlet surface 68 of the pivotable prism 65.

Both the reflecting surface 62 of the fixed prism 60 and the reflecting surface 67 of the pivotable prism 65 can reflect through total reflectance or on the basis of a reflecting coating. Contrary to FIG. 6, the light inlet surfaces 61, 66, the reflecting surfaces 62, 67, and the light outlet surfaces 63, 68 of the fixed prism 60 and of the pivotable prism 65 can each be curved in order to modify the convergence or divergence of the transmitted illuminating light bundle.

The light outlet surface 68 of the pivotable prism 65 is positioned opposite the light inlet surfaces 41 of pivotable light conductors 40 and is optically coupled with them. In particular, the light outlet surface 68 of the pivotable prism 65, contrary to the schematic depiction in FIG. 6, is soldered or joined with the light inlet surfaces 41 of the pivotable light conductors 40 by a transparent cement or a transparent welding. The ends downstream in the light path and the light outlet surface 42 of the light conductors 40, similarly as with the embodiment of FIGS. 2 and 3, are positioned in mountings 56 on the wall of a light shaft 24. In the example shown in FIG. 6, one of the two pivotable light conductors 40 comprises no curvature or only a low curvature, while the other light conductor comprises an essentially S-shaped curvature.

To pivot the illumination and viewing angle around the pivot axis 28, the pivotable prism 65 and the pivotable light conductors 40 are pivoted around the axis 28 together with the pivotable prism 82 in the observation beam path 80, the light shaft 24 and mountings 56. For this purpose a pivotable illumination and observation device can be provided, similar to the one described above with reference to FIG. 3. Similarly as with the pivotable light conductors described above with reference to FIGS. 2 and 3, the pivotable light conductors 40 of the embodiment of FIG. 6 can also comprise a bundle of lightwave conductors 44 each. To improve mechanical robustness, the lightwave conductors 44 of the pivotable light conductors 40 can be cemented, melded, or cast with one another or possibly with a pivotable illumination and observation device that is not shown in FIG. 6.

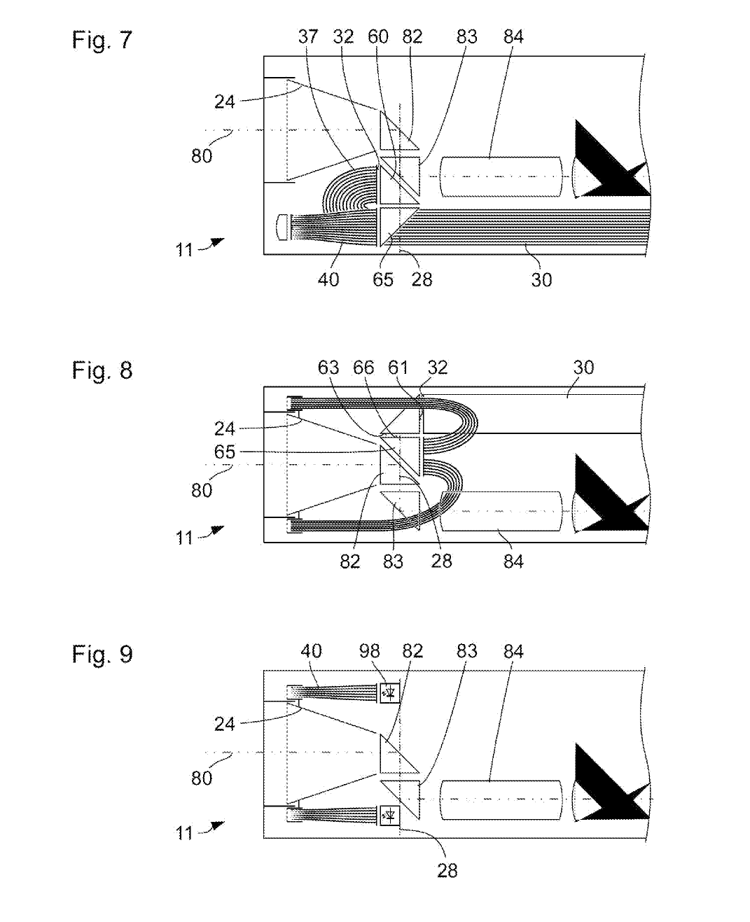

FIG. 7 shows a schematic depiction of another embodiment of the distal end 11 of the endoscope 10 described above with reference to FIG. 1. The embodiment in FIG. 7 resembles in some characteristics the embodiment described above with reference to FIG. 6. In particular, the observation beam path 80, with a pivotable prism 82, a fixed prism 83, and a rod lens device 84, corresponds to the observation beam path of the embodiments described above with reference to FIGS. 2, 3, and 6.

In addition, in the embodiment of FIG. 7 similarly as in the embodiment of FIG. 6, a fixed prism 60 and a pivotable prism 65 are foreseen in the illumination beam path. In the embodiment in FIG. 7, the fixed prism 60, however, is arranged in such a way that the reflecting surface of the fixed prism 60 of the illumination beam path and the reflecting surface of the fixed prism 83 of the observation beam path are in proximity to one another and are essentially in parallel arrangement. This allows an economy in structural space.

The fixed light conductor 30 comprises a curvature 37 of 180 degrees immediately upstream in the light path from its light outlet surface 32 and in this area in FIG. 7 is partly hidden behind the pivotable prism 65. The light outlet surface 32 of the fixed light conductor 30 is optically coupled with the light inlet surface 61 of the fixed prism 60 and can be connected with it, in particular, by soldering, cementing or by means of a transparent welding or cement.

The light outlet surface 68 of the pivotable prism 65 is optically coupled with the light inlet surface 41 of a pivotable light conductor 40, and in particular is soldered, cemented, or connected with it by means of a transparent solder or cement. The pivotable light conductor 40 includes, in particular, a bundle of lightwave conductors that are soldered, cemented, or cast together. The light outlet surface 42 of the pivotable light conductor 40 is coupled with a lens 58 to form the illuminating light beam. A lens of this type can also be provided in the embodiments of FIGS. 2, 3, 6, and the embodiments of FIGS. 8 and 9, to be described below, on the light outlet surfaces 42 of the pivotable light conductors 40.

In the embodiment in FIG. 7, therefore, the fixed prism 60 of the illumination beam path and the fixed prism 83 of the observation beam path are positioned between the pivotable prism 65 of the illumination beam path and the pivotable prism 82 of the observation beam path. If an illumination and observation device is provided similar to the one described above with reference to FIG. 3, the fixed prisms 60, 83 are positioned in a recess in the illumination and observation device.

Contrary to the depiction in FIG. 7, two or more pivotable light conductors 40 can be provided. The light outlet surfaces of the pivotable light conductors 40, similarly as with the embodiments described above with reference to FIGS. 2 through 6, can be positioned on the edge 25 or close to the edge 25 of the light shaft 24.

FIG. 8 shows a schematic depiction of another embodiment of the distal end 11 of the endoscope 10 described above with reference to FIG. 1. The embodiment in FIG. 8 resembles in a few characteristics the embodiments in FIGS. 2, 3, 6 and in particular the embodiment in FIG. 7.

The embodiment in FIG. 8 differs from the embodiment of FIG. 7 in particular in that two pivotable light conductors are foreseen whose light outlet surfaces 42 are positioned in mountings 56 on the edge 25 of the light shaft 24, similarly as with the embodiments of FIGS. 2, 3, and 6. In addition, it is not the fixed prisms 60, 83 of the illumination beam path and of the observation beam path but rather the pivotable prisms 65, 82 of the illumination beam path and of the observation beam path that are positioned close to one another. The pivotable prisms 65, 82 of the illumination beam path and of the observation beam path are positioned close to their reflecting surfaces and essentially parallel to one another and together form a compact, essentially quadrilateral pivotable component and/or are held in a pivotable illumination and observation device similarly to the one described above with reference to FIG. 3.

In the depiction in FIG. 8 a pivotable light conductor 40 is partially covered by the fixed prism 83 of the observation beam path 80 and the rod lens device 84, and another light conductor partly covers the fixed light conductor 30 and the fixed prism 60 of the illumination beam path. Contrary to the depiction in FIG. 8, the light conductors 40 can be positioned and curved as desired inside the physical borders applying for the light conductors 40.

Contrary to the depiction in FIG. 8, a light source, for example a light-emitting diode, can be positioned on the light inlet surface 66 of the pivotable prism 65 in the illumination beam path or close to this surface. In this case the fixed prism 60 and the fixed light conductor 30 can be dispensed with.

FIG. 9 shows a schematic depiction of another embodiment of the distal end 11 of the endoscope 10 described above with reference to FIG. 1. The embodiment in FIG. 9 resembles in a few characteristics the embodiments of FIGS. 2, 3, 6, and 8. The embodiment in FIG. 9 differs from the latter ones in particular in that in each case one light source 98 is coupled with the light inlet surface 41 of each pivotable light conductor 40. In the illustrated example the light conductors 40 are essentially straight. Contrary to the example shown in FIG. 9, the pivotable light conductors 40 can be curved.

The light sources 98 can be pivoted around the pivot axis 28 of the illumination and viewing angle. The pivotable light conductors 40 are therefore not deformed when the illumination and viewing angle pivots around the pivot axis 28. This reduces the risk of damage to the pivotable light conductors 40 and increases their useful life. Alternatively, for example, only parabolically or elliptically formed reflectors can pivot with the illumination and viewing angle in whose focal points the fixed light sources 98 are positioned. The light sources 98 are, for example, light-emitting diodes or fluorescent or phosphorescent bodies that can be excited by means of a laser beam transmitted by a lightwave conductor.

A few characteristics of the embodiments described above with reference to FIGS. 2 through 9 can be combined in other ways than as shown. For example, in all embodiments it is possible to foresee lenses for forming the illuminating light beam on the light outlet surfaces 42 of the pivotable light conductors 40, as is the case with the embodiment in FIG. 7. In addition, in each embodiment of FIGS. 6 through 9 the light shaft 24 and its edge 25, as well as in some cases the mounting or mountings 56, can be configured according to one of the variants described above with reference to FIG. 4. The light inlet surface 41 of the pivotable light conductor or conductors in all embodiments can be configured according to one of the variants described above with reference to FIG. 5.

In the embodiments described above with reference to the drawings, the viewing angle and the angle of illumination can be pivoted around a common axis. For this purpose, a pivotable illumination and observation device can be provided, in particular, as is described above with reference to FIG. 3. Alternatively the viewing angle and angle of illumination can be pivotable around two different axes that, in particular, are parallel.

In particular when the pivotable illumination and observation device, described above with reference to FIG. 3, is provided, the viewing angle and angle of illumination can be pivoted together. Here the viewing angle and angle of illumination are always parallel or always differ from one another by a constant angle. Alternatively the viewing angle and the angle of illumination can be independently pivotable, or there is an adjustable angle between the viewing angle and the angle of illumination, in particular inside predetermined boundaries. This can make possible, for example, a complete illumination of the visual field when objects are at short distances.

All embodiments and their variants show that light conductors 40—which are pivotable together with their light inlet surfaces 41 and their light outlet surfaces 42 around the pivot axis 28 of the illumination and viewing angle and upon pivoting, in particular, are not deformed—create additional free spaces in the configuration of the distal end 11 of an endoscope and make possible novel, compact, and advantageous configurations.

The light outlet surfaces 42 of the pivotable light conductors can be positioned as a rule in the immediate vicinity of a window component through which the illuminating light exits. Thus the illuminating light remains bundled on a small cross-section, namely the cross-section of the light conductor or light conductors, until immediately before the window component. This allows for a compact, narrow window component and encourages a reduction of the endoscope size.

The embodiments in FIGS. 2 through 4 and 6 through 9 each comprise diaphragms 27 for optical severing of the illumination beam path or illumination beam paths from the observation beam path. In all embodiments, the diaphragms 27 can each border on a pass-through or common window component 20 (compare FIG. 1), such that both illuminating light exiting at the distal end 11 of the endoscope 10 and observation light entering the distal end 11 of the endoscope 10 flow through the pass-through or common window component. The space in which illuminating light can spread out is thus only separated or optically isolated by a wall up to the inner surface of the window component 20 from the observation beam path.

Alternatively, several separate window components can be provided so that a first window component is positioned only in the observation beam path and one or more second window components are positioned exclusively in the illumination beam path. The first window component and the second window component or components are separate components. In particular, the first window component is separated in each case by one separating wall or diaphragm 27 or their edge area, so that the separating wall or diaphragm 27 is not permeable to light. The separating wall or diaphragm 27, in particular, is configured to separate the edge area in which illuminating light spreads from the edge area in which the video camera 80 is positioned, completely and above all in light impermeable manner, all the way to the outer surface of the endoscope 10. This can prevent a switching of illuminating light, which is dispersed in the second window component or in its surface, into the video camera 80.

In each of the embodiments described here, a pass-through window component or several separate window components can be provided. In addition, a pass-through or common window component with a light-absorbent separating layer can be provided that prevents an undesired direct switching of illuminating light into the observation beam path. Such a separating layer can be generated, for example, by ions implanted or otherwise locally incorporated into the material of the window component.

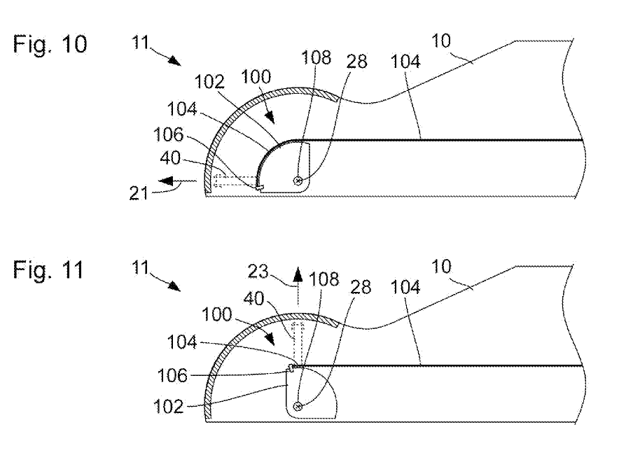

FIGS. 10 and 11 show schematic depictions of a device 100 for selecting the angle of illumination and the angle of observation 21, 23 of an endoscope 10. The planes of projection of FIGS. 10 and 11 are parallel to the planes of projection of FIGS. 1 and 5, parallel to the longitudinal axis 18 of the endoscope 10 (compare FIG. 1), perpendicular to the pivot axis 28 (compare FIGS. 2 through 9), and perpendicular to the planes of projection of FIGS. 2 through 4 and 6 through 9. The distal end 11 of the endoscope 10 is constituted in FIGS. 10 and 11 in two different illumination and observation devices 21, 23. Pivotable light conductors 40 with mountings near their light outlet surfaces are indicated by way of example in dotted lines. The device 100, for example, is configured to pivot the movable illumination and observation device 90 that is described above with reference to FIG. 3. In addition, the device 100 can be configured to pivot the pivotable prisms 65, 82, the light shaft 24 and/or the pivotable light conductors 40 by others of the embodiments described above with reference to FIGS. 2 through 9.

The device 100 includes a disc device 102, which is fastened onto a shaft 108. The disc device 102 can be pivoted with the shaft 108 around the pivot axis 28 that is also shown in FIGS. 1 through 9. A pivot movement of the disc device 102 results, for example, in a corresponding pivot movement of the illumination and observation device 90 described above with reference to FIG. 3 and the illumination and observation device 21, 23. The disc device 102 comprises a round-arched edge portion of which the center point of curvature is situated on the pivot axis 28.

In the shaft of the endoscope 10, a belt device 104 extends from the proximal end 12 of the endoscope 10 to the distal end 11 of the endoscope 10. The belt device 104 includes, in particular, a wire or a band of metal or plastic and comprises a low extension elasticity. The distal end of the belt device 104 is mechanically connected with the disc device 102 by means of a fastening device 106.

It can be recognized from a comparison of FIGS. 10 and 11 that a movement of the belt device 104 in the longitudinal direction of the shaft of the endoscope 10 causes a pivot movement of the disc device 102, pivotable light conductor 40, and illumination device 21, 23. In a pivot movement of the disc device 102 in clockwise direction, a distal area of the belt device 104 is removed from the round-arched edge area of the disc device 102. In a counterclockwise pivot movement of the disc device 102, a distal area of the belt device 104 is imposed or reeled onto the round-arched edge area.

The belt device 104, in particular, is rigid only under tensile stress and cannot transmit pushing forces. To make possible nevertheless a pivot movement of the disc device 102, pivotable light conductor 40, and illumination and observation device 21, 23 in counterclockwise direction, it is necessary to provide a spring or other elastic element on the distal end 11 of the endoscope 10 that is not shown in FIGS. 10 and 11. This spring or other elastic element pretenses the disc device 102 and the pivotable light conductor 40 in the direction toward the position shown in FIG. 10.

1. An endoscope with adjustable viewing angle with:
a light conductor with a light inlet surface and a light outlet surface to transmit illuminating light from the light inlet surface to the light outlet surface of the light conductor,
such that the light conductor can be pivoted with the light inlet surface and light outlet surface in relation to the endoscope around a pivot axis that is perpendicular to the longitudinal axis of the shaft of the endoscope.

2. The endoscope according to claim 1, wherein the pivotable light conductor is rigid.

3. The endoscope according to claim 1, wherein the pivotable light conductor includes a bundle of lightwave conductors that are cemented, melded, or cast together in order to stiffen the bundle.

4. The endoscope according to claim 1, in addition with:
a pivotable reflecting surface to divert illuminating light from a direction parallel to the pivot axis of the pivotable light conductor to the light inlet surface of the pivotable light conductor, such that the pivotable reflecting surface is designed and configured in order to be pivoted together with the pivotable light conductor.

5. The endoscope according to claim 4, wherein the pivotable reflecting surface is a reflecting surface of a pivotable prism with a light inlet surface and a light outlet surface, and
the light inlet surface of the pivotable light conductor is joined with the light outlet surface of the pivotable prism.

6. The endoscope according to claim 1, wherein the pivotable light conductor comprises a curvature.

7. The endoscope according to claim 6, wherein the pivotable light conductor is positioned on the light inlet surface of the pivotable light conductor in a direction parallel to the pivot axis of the pivotable light conductor.

8. The endoscope according to claim 7, wherein the light inlet surface of the pivotable light conductor is circular or circular-ring-shaped.

9. The endoscope according to claim 1, in addition with:
a fixed light conductor to transmit illuminating light to the distal end of the endoscope, such that the fixed light conductor comprises a curvature close to its light outlet surface.

10. The endoscope according to claim 9, in addition with:
a light shaft that can pivot around the pivot axis of the viewing angle to optically separate the observation beam path from the illumination beam path,
such that the light outlet surface of the pivotable light conductor is positioned outside on the light shaft.
